(19) United States
(12) Patent Application Publication
(10) Pub. No.: US 2023/0185360 A1
(43) Pub. Date: Jun. 15, 2023

(54) DATA PROCESSING PLATFORM FOR INDIVIDUAL USE

(71) Applicant: Logitech Europe S.A., Lausanne (CH)

(72) Inventors: John SANGIOVANNI, Seattle, WA (US); Jared Andrew MESSENGER, Sunnyvale, CA (US); Michele Lee MCMULLEN, Seattle, WA (US)

(21) Appl. No.: 17/548,322

(22) Filed: Dec. 10, 2021

(51) Int. Cl. G06F 3/01; A61B 5/16; A61B 5/00

(52) U.S. Cl. CPC G06F 3/011; A61B 5/165; A61B 5/743; A61B 5/7282; A61B 5/7475; G06F 2203/011

(57) ABSTRACT

Embodiments herein provide methods, systems, suitable for the analysis of both complex and conventional data pertaining to an individual user or human activity that is received in real-time and/or in batches from a variety of data source types, to identify and produce results that benefit the individual user. One general aspect includes a method comprising: receiving input data from a plurality of peripheral devices, e.g., one or more interface devices for a user device; analyzing the input data to generate a plurality of data analysis streams of time-series data relating to a user of for a first period of time; generating a plurality of user or system generated insight based time tags for second periods of time within the first period of time; and generating a dashboard for display to the user. The dashboard may include a plurality of charts, each representing a data analysis stream over the first time period, and a plurality of time-tag representations extending across the plurality of charts at the second time periods.

### BACKGROUND

Due to their ubiquity, consumer electronic devices can generate a tremendous amount of data pertaining to all aspects of a user’s daily life. Big data analytics have leveraged access to collective data received through the Internet of Things to design and market products and services. Consumers are inundated with products promising life improvements through the individual tracking of aspects of a user’s activities, health, or wellbeing, yet demand for such products continues unabated. Unfortunately, such individual electronic devices are unable to generate the type of insights that might be realized from a system capable of receiving and analyzing the volume, velocity, and variety of data generated from the user’s daily engagement with multiple electronic devices.

In the current electronic age, there is also an interest and a need for users of various connected electronic devices to prevent their personal attributes or personal information from being disseminated to a third party that may use the user’s information in such a way that it will damage the user’s reputation in society, monetary worth, self-worth or other user attributes. Moreover, patient privacy laws and consumer privacy laws have been enacted that can make various entities and other third parties liable for the use of and/or dissemination of sensitive user information, such as the Health Insurance Portability and Accountability Act (HIPAA) laws, European Union’s General Data Protection Regulation (GDPR) and California’s California Consumer Privacy Act (CCPA). Therefore, individuals and entities that develop products that receive and use user data need ways to receive and use information relating to a user’s activities, health, or wellbeing so that the information can be used to provide insights that improve aspects of an activity that the user is performing without being concerned about violating privacy laws and also assuring a user that their personal information received by the product will not be delivered to or used by a third party.

Accordingly, there is a need for a system that solves the problems described above.

### SUMMARY

Embodiments herein provide methods, systems, and devices suitable for the analysis of both complex and conventional data pertaining to an individual user or human activity that is received in real-time and/or in batches from a variety of data source types to identify and produce results that benefit the individual user. A system of one or more computers can be configured to perform particular operations or actions of the embodiments by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

One general aspect includes a computer-implemented method for improving user performance. The computer-implemented method includes: a) receiving input data from a plurality of peripheral devices, the plurality of peripheral devices may include one or more interface devices that are integrated with or connected to a user device; (b) analyzing the input data to generate signal stream information that may include a plurality of data analysis streams, each of the plurality of data analysis streams may include time-series results data for a first period of time relating to a user; (c) generating a plurality of time tags corresponding to second periods of time within the first period of time, where one or more of the plurality of time tags are based on insights relating to the user; and (d) generating a dashboard for display to the user. The dashboard may include a plurality of data analysis stream charts aligned by a common time axis, each chart graphically representing the time-series data over the first time period for a respective one of the plurality of data analysis streams, and a plurality of time-tag representations extending across the plurality of data analysis stream charts at the second time periods.

One general aspect includes a computer-implemented method for improving the performance of one or more user activities. The computer-implemented method also includes (a) receiving, by a user device, time-series input data generated by a user’s interactions with the user device through a plurality of interface devices; (b) analyzing the time-series input data to generate a plurality of data analysis streams, each of the data analysis streams containing time-series results data relating to the user for a first period of time; (c) receiving, by use of a user interface application, user insights describing one or more events, ambient conditions, behaviors, mental states, and/or physical states experienced by the user at one or more second periods of time within the first period of time. The method also includes (d) generating one or more system insights, may include: (i)determining that there are changes in at least two of the data analysis streams that happened concurrently or proximately in time and, based on the changes, determining that an event has occurred; or (ii) determining a relationship between one or more of the data analysis streams and a user insight, where generating the one or more system insights may include applying one or more rules stored in memory; and (e) generating a dashboard for display to the user. The dashboard may include graphical representations of one or more of the data analysis streams, the user insights, and the system insights.

One general aspect of the disclosure provided herein includes a system for improving user performance in one or more activities. The system also includes a plurality of interface devices communicatively coupled to and/or integrated with a user device, where one or more of the plurality of interface devices may include a keyboard device, a camera device, a mouse device, a microphone, or a gaming controller; one or more applications stored in memory, where the one or more applications are configured to: (a) receive time-series input data from the plurality of interface devices; (b) analyze the time-series input data to generate a plurality of data analysis streams, where one or more of the data analysis streams contain time-series results data characterizing an aspect of the user’s performance of an activity on the user device and one or more of the data analysis streams contain time-series results data characterizing an aspect of the user’s behavior during performance of the activity; (c) receive user insights describing one or more events, ambient conditions, behaviors, mental states, and/or physical states experienced by the user during performance of the activity; and (d) generate a dashboard for display to a user. The dashboard may include graphical representations of one or more of the data analysis streams and the user insights.

One general aspect includes a computer-implemented platform for improving user performance. The computer-implemented platform also includes a) receiving input data from a plurality of peripheral devices that are integrated with or in communication with a user device, where the plurality of peripheral devices are selected from a group may include interface devices, personal devices, sensors, and virtual devices may include remotely executed non-platform software; (b) analyzing the input data to generate signal stream information may include a plurality of data analysis streams, each of the plurality of data analysis streams may include time-series results data for a first period of time relating to a user; (c) generating a plurality of time tags corresponding to second periods of time within the first period of time, where one or more of the plurality of time tags are based on insights relating to the user; and (d) generating a dashboard for display. The dashboard may include: a plurality of data analysis stream charts aligned by a common time axis, each chart graphically representing the time-series results data over the first period of time for a respective one of the plurality of data analysis streams; and a plurality of time-tag representations at the second periods of time.

One general aspect includes a computer-implemented platform for improving user performance, including one or more platform applications stored in memory, where the one or more platform applications are configured to: a) receive time-series data relating to a user during a first period of time, where the time-series data is received from a plurality of peripheral devices that are integrated with or in communication with a user device, where the plurality of peripheral devices are selected from a group may include interface devices, personal devices, sensors, and virtual devices may include locally or remotely executed non-platform software; (b) generate a plurality of time tags corresponding to second periods of time within the first period of time, where one or more of the plurality of time tags are based on insights relating to the user; and (c) generate a dashboard for display. The dashboard may include signal stream information and a plurality of time-tag representations at the second periods of time, where the signal stream information is represented in a plurality of data analysis stream charts aligned by a common time axis, and the signal stream information may include: (i) one or more data analysis streams received in the time-series data; (ii) one or more data analysis streams generated by an analysis of the time-series data received from the plurality of peripheral devices; or (iii) a combination of (i) and (ii).

Other embodiments of the above aspects of the disclosure include corresponding computer systems, apparatus, and computer programs recorded on one or more computer storage devices, each configured to perform the actions of the methods.

### BRIEF DESCRIPTION OF THE DRAWINGS

So that the manner in which the above-recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, may admit to other equally effective embodiments.

FIG. 1 is a block diagram of a system architecture, according to one embodiment.

FIG. 2 is a block diagram of a developer platform, according to one embodiment.

FIG. 3 is a block diagram illustrating an example configuration of the system, according to one embodiment.

FIG. 4 is a block diagram illustrating an example configuration of the system, according to one embodiment.

FIG. 5 is a block diagram illustrating an example configuration of the system, according to one embodiment.

FIG. 6 is a screen shot of a first view of a dashboard of the user interface generated using the methods described herein, according to one embodiment.

FIG. 7 is a screen shot of a second view of the dashboard of FIG. 6, according to one embodiment.

FIGS. 8A-8D are screen shots showing various features of the user interface, according to one or more embodiments.

FIG. 9 is a block diagram of a device configured to implement the systems described herein, according to one embodiment.

FIG. 10 is a block diagram of a device configured to implement the systems described herein, according to one embodiment.

FIG. 11 is a flow diagram of a method that may be performed using the systems described herein, according to one embodiment.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

Embodiments of the disclosure provided herein include methods, systems, and devices that actively filter sensitive information from one or more data streams that, based on the receipt and analysis of the filtered information, are used to improve an individual user’s performance of an activity. In some aspects of the disclosure, the analysis of the filtered information includes using one or more algorithms that are configured to automatically identify and characterize the user’s performance of the activity and factors affecting the user’s performance of the activity. One or more algorithms may be further configured to provide recommendations that can be implemented by the user to improve the efficient and effective performance of the activity.

The methods, systems, and devices described herein provide a system platform suitable for the analysis of both complex and conventional data pertaining to an individual user or human activity that is received in real-time and/or in batches from a variety of data source types to identify and produce results that benefit the individual user. In some embodiments, the disclosed system platform includes one or more software applications executed on a device routinely used by the user to perform computer-related activities. In other embodiments, the system platform includes one or more software applications executed on a peripheral device disposed in wired or wireless communication with a device associated with the individual user or user device. Generally, data is received by the platform from a plurality of data sources that include peripheral devices and software applications other than the software applications used to execute the functions of the system platform.

As used herein, “user devices” may include personal computing devices, such as laptop or desktop computers, mobile computing devices such as smartphones or tablets, and gaming devices, such as gaming consoles or handheld devices. “Peripheral devices” include any electronic device or hardware which is integrated with or is configured to establish communication with the user device to transfer data thereto or therefrom, such as interface devices, personal electronic devices, and sensors and environmental control systems. “Interface devices” generally include electronic devices and hardware that enable a user to interact with the user device and may include input devices, such as keyboards, mice, cameras, microphones, and gaming controllers, and output devices, such as display screens and audio speakers. “Personal devices” as referred to herein generally include electronic devices configured to operate independently from the user device that can be connected to the user device in order to transfer data, such as medical devices, wearable devices, smartphones, and tablets. “Sensors” and “environmental control systems” generally refer to electronic devices located in the user’s environment that may be used to measure and/or control ambient conditions, such as air quality sensors, noise sensors, and thermostats.

Herein, software applications used to execute one or more functions of the platform are generally referred to as “system applications,” “platform applications,” “system software,” “platform programs,” or may have no designation. Software applications that can be used as a source of data and that have a purpose other than to execute functions of the system platform are generally referred to as “non-platform software,” “non-platform applications,” or “non-platform software applications.” In some embodiments, the non-platform software, such as a calendaring application, is stored and executed locally, e.g., on a user device or a personal device disposed in communication with a user device. In some embodiments, the non-platform software, such as music stream services (Apple Music®, Spotify®, SoundCloud®, Prime Music Deezer®, Pandora®, etc.) may be stored and executed remotely, e.g., via the Internet. In those embodiments, the remote non-platform software may be referred to as “virtual device(s),” or “peripheral virtual device(s).” Such terms are not intended to be limiting however, as it is contemplated that one or more non-platform software applications or virtual devices may also be configured to perform one or more functions of the system platform, such as described in the examples below.

Data generated through the use of electronic devices and non-platform software, or derived therefrom, may be referred to as “time-series input data,” “input data,” “signal data,” “signal input data,” “input signal,” “signal input,” “event data,” “input events,” “event streams,” “filtered signal data,” “filtered input data,” “filtered data,” “filtered event data,” “analysis results,” “signal analysis results,” “analysis data,” “analysis data streams,” “signal stream data,” “signal stream information,” or the like. Data generated from user input and observations may be referred to as “user input” or “user insights.” In some embodiments, the system platform is configured to infer events from the analyzed input data, such as by use of a machine-learning artificial intelligence (Al) algorithm. The inferred events may be referred to as “system insights” or machine learning “(ML) insights.” User and systems-generated insights are typically time-indexed to a discrete-time or time period and may be referred to herein as “time tags.”

Typically, the data received by the system platform includes a combination of time-series data generated by a plurality of peripheral devices and/or non-platform software, and time-indexed observations actively collected from the user (user insights). Generally, the input data contains passively collected data, i.e., generated without direct interaction between the user and the system platform, such as data generated from the routine use of peripheral devices and/or non-platform software in communication with the system platform. For example, input data may be received from the user’s interaction with one or more peripheral interface devices (e.g., keyboard, mouse, gaming controller, camera, microphone), received from peripheral sensors passively monitoring the user’s environment (e.g., air quality sensors, ambient light sensors, temperature sensors), received from personal peripheral devices used to track health and activity-related information (e.g., biometric devices, activity trackers), generated by the user’s routine use of virtual peripheral devices, (e.g., Pandora®, Spotify®), or generated by the routine use of locally executed non-platform software (e.g., calendaring applications). Generally, the input data is used by the system platform to characterize aspects of the user’s activities, behavior, health, wellbeing, and surroundings.

Unlike the passively collected input data, generating user insights requires the active engagement of the user with the system platform. For example, collecting user insights may include receiving user observations regarding the user’s mental or physical state (e.g., “in the zone,” “feeling focused,” “feeling fatigued”) or observations into otherwise untracked events and ambient factors (e.g., “consumed a cup of coffee,” “worked with my cat on my lap”). In some embodiments, the platform is configured to identify relationships between the analysis results and the user insights through an iterative learning process that can be performed by one or more of the platform applications.

Through the learning process, the system platform may be taught to generate system insights on otherwise unmeasured or untracked factors or events that indicate or affect the user’s performance, health, and wellbeing, such as by use of a machine-learning artificial intelligence (Al) algorithm. For example, an Al algorithm may be used to infer events from changes within one or more of the time-series data analysis results, predict user performance based on learned user behaviors, or correlate analysis results to input data not otherwise tracked.

In some embodiments, the methods include receiving time-series input data from electronic devices and non-platform software routinely used by a user (e.g., personal computers, gaming consoles, computer and gaming peripherals, i.e., interface devices, personal devices, environmental control systems and sensors, calendaring applications, gaming applications, music subscription services), filtering identifying information from the input data to provide privacy-filtered data, and analyzing the filtered data to generate signal analysis results that characterize aspects of the user’s performance, health, wellbeing, and surroundings.

In some embodiments, the input data is received, filtered, and analyzed in real-time, and the signal analysis results are generated periodically, e.g., at one-minute intervals, to provide time-series analysis results, herein a signal stream information. In some embodiments, generating the signal analysis results includes generating feedback for the user based on a metric having a positive or negative association with the analyzed input data. For example, the feedback may be a score or a rating that may be used by the user to gauge and track improvements in their behavior, health, wellbeing, or surroundings over time. Typically, the feedback generated by one or more of the platform applications, e.g., a signal analysis application, is periodically published with the analysis results as part of the signal stream information.

The methods may include determining relationships between the signal analysis results and user and system-generated insights, and based on the determined relationships, recommending actions that the user can take to achieve healthier work habits. In some embodiments, the methods include presenting the signal analysis results, feedback scores, user and system-generated insights, and recommended actions to the user, e.g., by use of a comprehensive dashboard generated by one or more platform applications, e.g., a user interface application.

The dashboard aids the user in discovering the relationships between performance, health, wellbeing, and ambient factors using objective data generated from the routine use of electronic devices in daily activities. For example, the data may be generated using electronic devices configured to perform computer-related work tasks, computer-related gaming activities, monitor aspects of the user’s health and/or activity, or monitor ambient conditions in the user’s environment. In some embodiments, the dashboard is configured to concurrently display a plurality of graphs representing the signal stream information, herein signal stream graphs, wherein at least two of the signal stream graphs contain signal stream information generated using input data received from different data sources.

In one example, as will be discussed further below, the plurality of signal stream graphs may include hardware-related signal stream information (e.g., keyboard keystroke rate, typing error rate, mouse movement rate), environment signal stream information (e.g., environment CO₂ level, room temperature, ambient light amount, noise level), audio signal stream information (e.g., audio source sound volume, song type being played, music’s beats per minute), biometric signal stream information (e.g., user’s heart rate, user’s blood sugar level, user’s pulse oximetry, user’s stress level, user’s respiration rate, eye blink rate), and non-platform software application signal stream information (e.g., calendar data or gaming-related statistics such as actions per minute (APM) and gaming critical hit ratio). The plurality of signal stream graphs may be vertically stacked and share a common time axis to provide a timeline view that allows the user to visualize trends and changes in the represented signal stream information over time, as well as relationships between the signal stream information represented in the different signal stream graphs. In one example, as will be discussed below, a comparison of the information found in two or more signal stream graphs can be used to determine how various factors can affect a user’s performance. As an example, a software application (e.g., one of the platform applications 912) can determine that there is a correlation between a high typing error rate and a high CO₂ level in the user’s environment and thus provide a suggestion to the user to open a window or, by use of various automation hardware (e.g., building automation systems, smart home hardware, etc.), automatically increase the HVAC’s turn-over of the user’s environment or automatically open a window.

In some embodiments, vertical bars or columns representing the user and system-generated insights for discrete-time periods, referred to herein as “time tags,” are overlaid across the plurality of signal stream graphs so that the user may better visualize the intersections therebetween. In some configurations, user and system-generated insights can include information regarding an event that impacts one or more of the plurality of signal stream graphs or an event inferred from an analysis of information in one or more of the plurality of signal stream graphs. An example of some user-generated insights can include inputs provided by a user such as “away from the computer,” “consumed a cup of coffee,” “feeling sleepy,” “have a migraine,” “took a nap,” “the cat came to visit,” or had a “stressful meeting” or other useful insight(s) based on the current or past experience of the user. Examples of some system-generated insights can include the “CO₂ level is at an undesirable level”, “room temperature is at an undesirable level,” “user-worked 4 hours straight”, “reminder to drink water,” or other useful insight(s) based on the system’s analysis of information contained in the plurality of signal stream graphs.

Example systems that may be used to perform the methods are described below and generally include a platform for managing data privacy and security, device access, integration of third-party applications, a plurality of signal analysis applications for analyzing time-series input data received from a plurality of data sources, and a user interface for presenting the comprehensive dashboard to the user. It should be noted that although the illustrative examples of the methods and systems provided herein are generally directed to performance at computer-based work and/or gaming activities, embodiments are not so limited, as it is contemplated that the systems and methods may be used to optimize individual performance within any desired endeavor.

### SYSTEM OVERVIEW

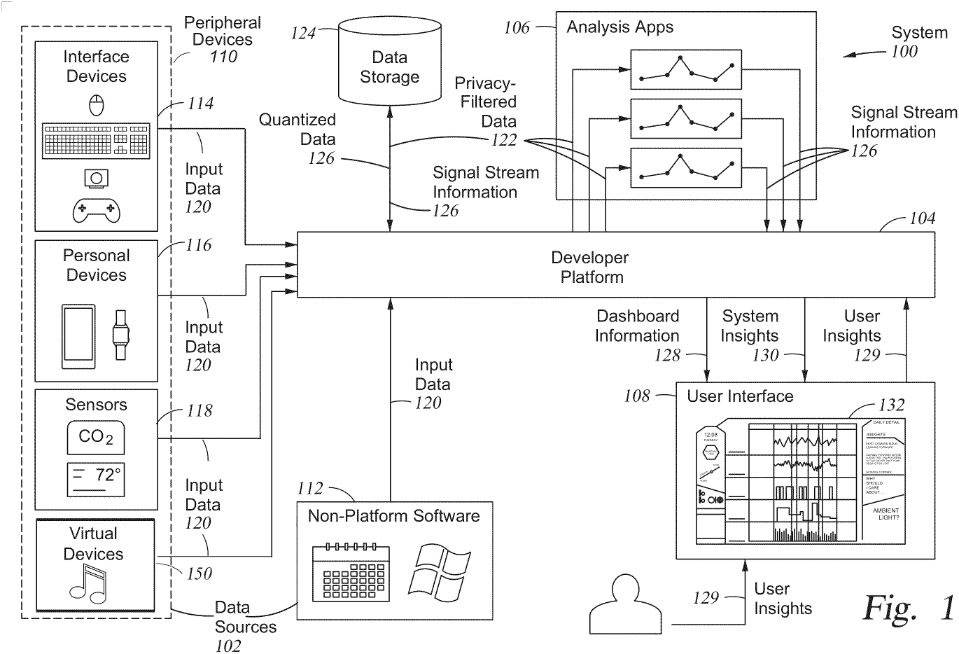

FIG. 1 provides a high-level overview of a system that may be used to perform the methods described herein, according to one embodiment. Here, the system 100 is configured to receive and analyze data from a plurality of data sources 102 and includes a developer platform 104, a plurality of signal analysis applications 106, and a user interface 108. The plurality of data sources 102 generally include, but are not limited to, a combination of peripheral devices 110 and local non-platform software 112 that are routinely employed by a user to accomplish tasks, monitor health and wellbeing, and measure and/or control ambient conditions in the user’s environment. Examples of peripheral devices 110 include interface devices 114 used to input data to a user device (e.g., keyboards, mice, cameras, gaming controllers), personal devices 116 configured for independent operation which can be connected to a user device (e.g., tablets, biometric wearables, smartphones), sensors 118 used to measure and/or control ambient conditions (e.g., thermostats, carbon dioxide sensors, ambient light sensors, noise sensors), and remotely executed non-platform software, herein virtual devices 152 (e.g., cloud-based media content providers and gaming stat tracking services).

The developer platform 104 is generally configured to manage various aspects of the collection, privacy control, security control, and analysis of input data 120 received from the plurality of data sources 102 as well as communication with and/or between the data sources 102, the signal analysis applications 106, and the user interface 108. In some embodiments, the developer platform 104 is configured to receive input data 120 from each of the data sources 102, remove identifiable information from the input data 120 to generate privacy-filtered data 122, and provide the privacy-filtered data 122 to one or more of the plurality of signal analysis applications 106. Depending on the type of data source and input data type, the developer platform 104 may securely store the privacy-filtered data 122 in data storage 124 and/or facilitate access to the privacy-filtered data 122 by the signal analysis applications 106 in real-time.

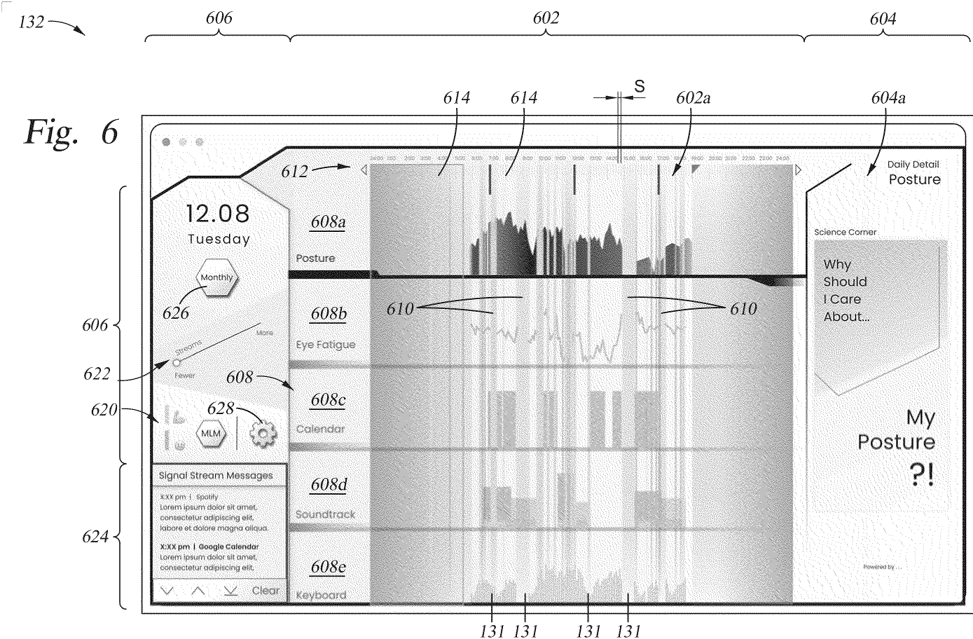

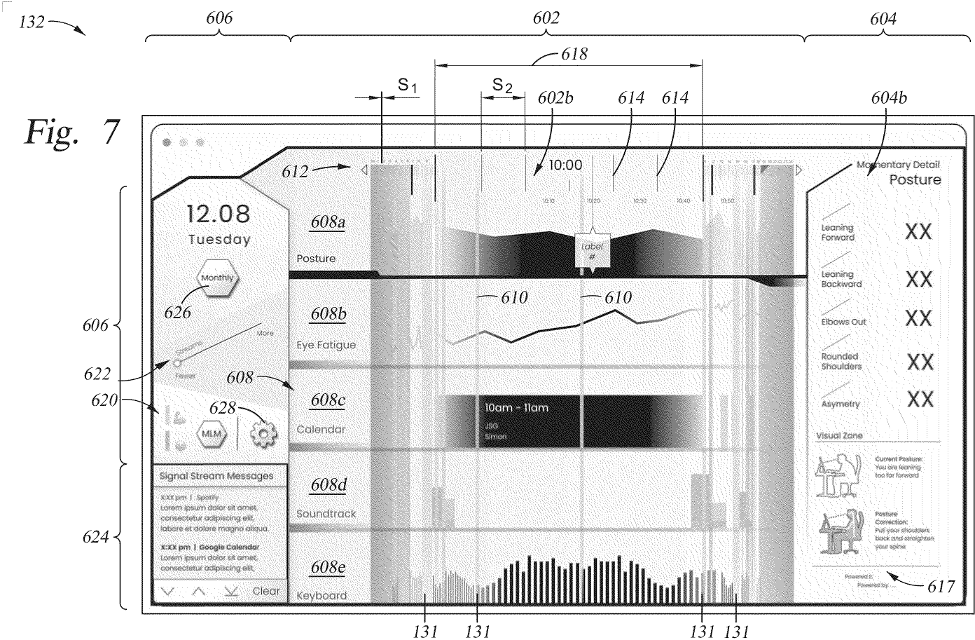

In some embodiments, one or more of the signal analysis applications 106 are configured to receive one or more streams of privacy-filtered data 122 from the developer platform 104, perform one or more comparisons or calculations on the privacy-filtered data 122 to generate analysis results, e.g., signal stream information 126, and periodically publish the signal stream information 126 to the developer platform 104 as one or more time-series data analysis streams. In some embodiments, the signal stream information 126 includes feedback scores periodically generated by the signal analysis applications 106 based on the analysis results. In some embodiments, the developer platform 104 may generate feedback based on the signal stream information 126, and communicate the signal stream information 126 and the feedback scores to the user interface 108 for display in a dashboard 132 that may be formed on a display device. In some embodiments, the signal analysis applications 106, the signal stream information 126, and graphical representations of the signal stream information, e.g., the signal stream graphs 608 described below in relation to FIGS. 6-7, are referred to collectively as signal streams.

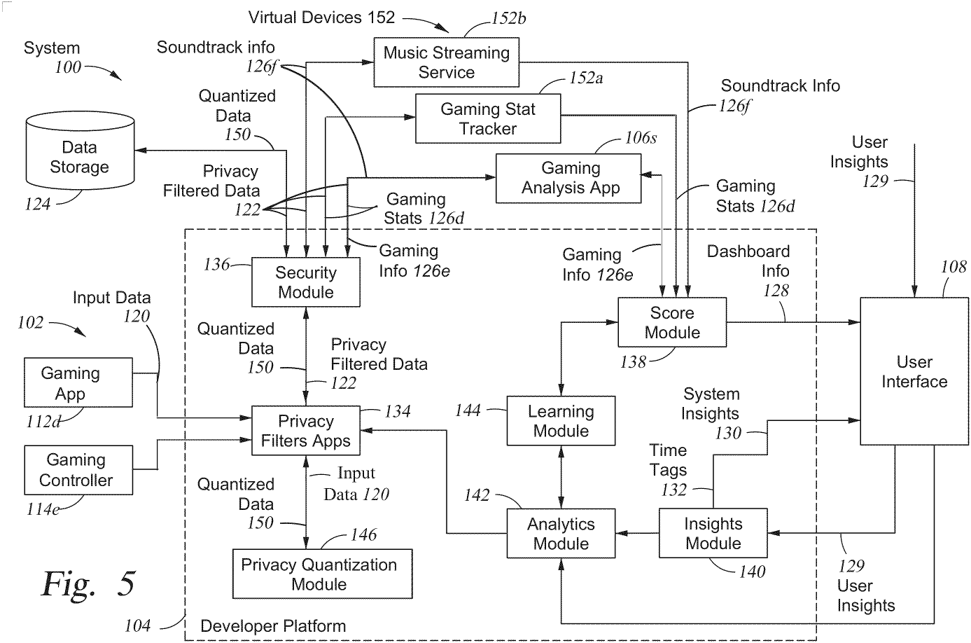

In some embodiments, each of the signal analysis applications 106 communicate directly with the developer platform 104 via a software development kit (SDK) provided by the developer. The plurality of signal analysis applications 106 may include developer applications, third-party applications, user-generated applications, or combinations thereof. In some embodiments, one or more of the signal analysis applications 106 are JavaScript applications that operate and communicate with the developer platform 104 in a virtual environment. In some embodiments, one or more of the data sources 102 may be configured to generate and provide signal analysis information 126 directly to the developer platform 104, such as described in relation to FIG. 5.

### EXAMPLE DEVELOPER PLATFORM

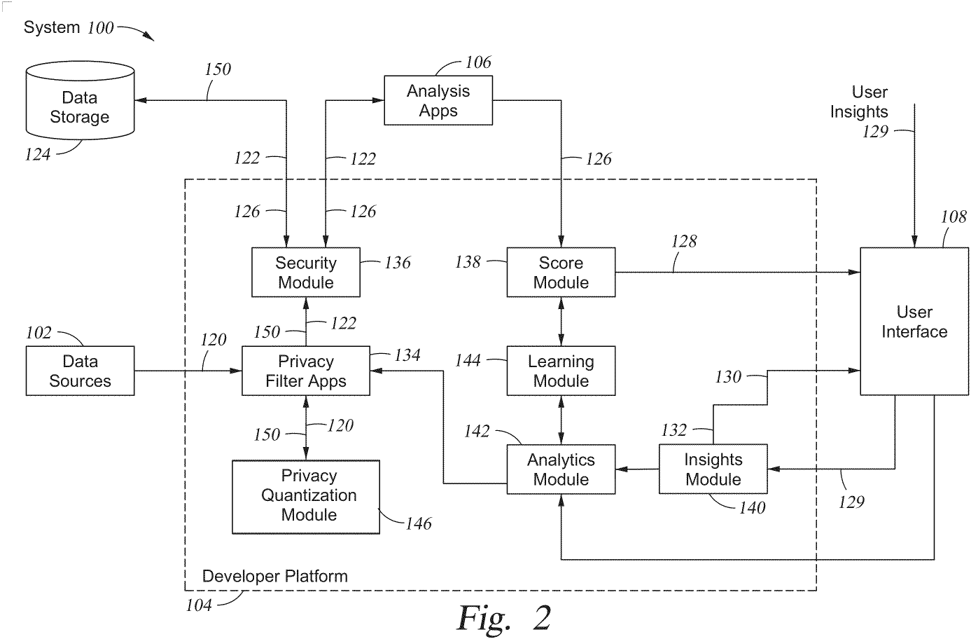

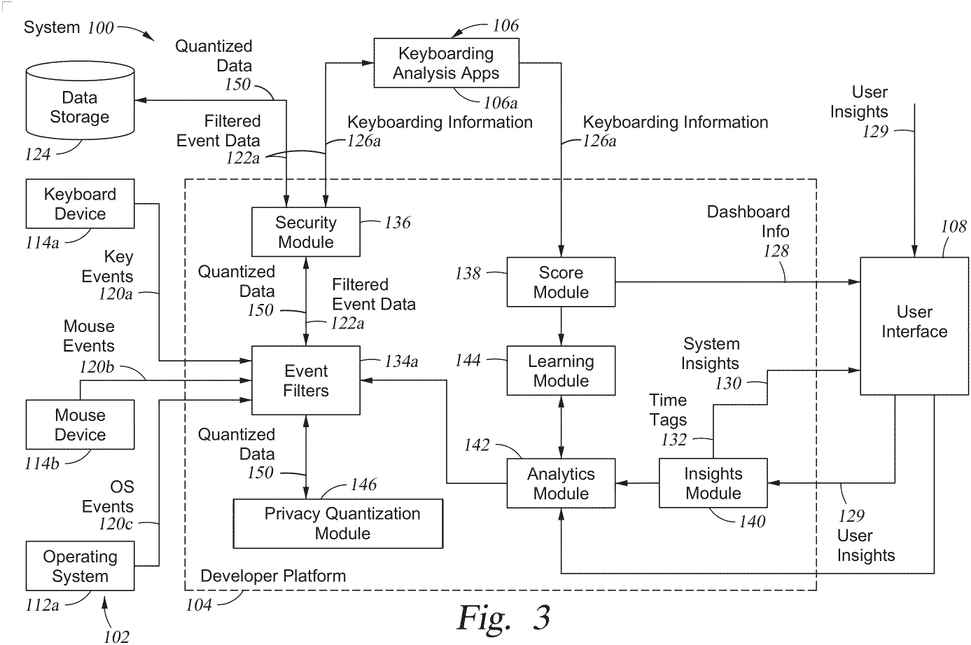

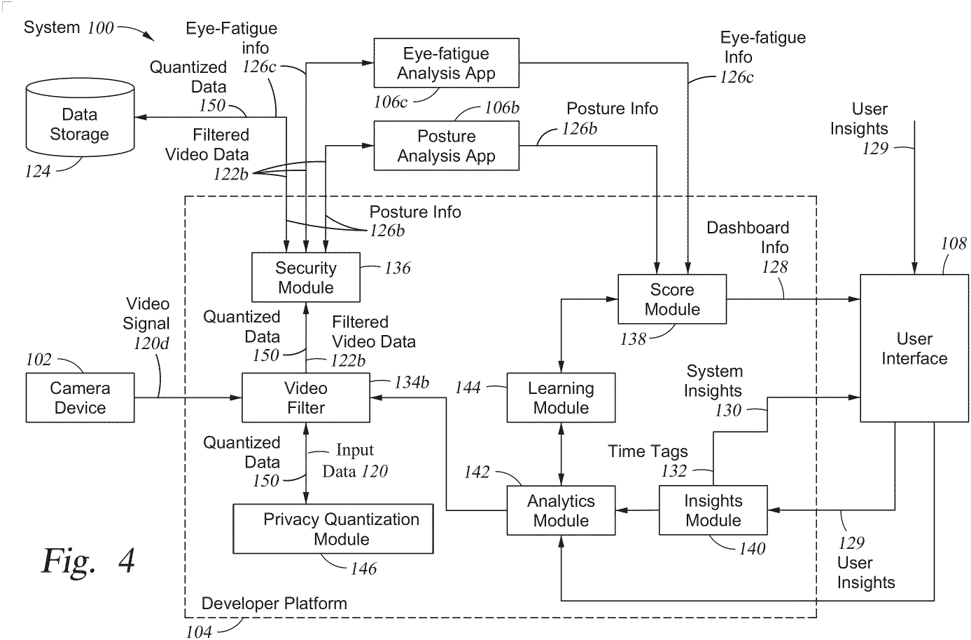

FIG. 2 is a block diagram of the developer platform 104, according to one embodiment. FIGS. 3-4 illustrate example configurations of the developer platform. As noted above, the developer platform 104 is configured to manage the collection, privacy control, and security control of data received from the plurality of data sources 102, facilitate communication between the data sources 102, the signal analysis applications 106, and the user interface 108, identify relationships between signal stream information 126 generated by one or more of the signal analysis applications 106 and user and system-generated insights 129, 130, and recommend actions that a user can take to achieve healthier work habits and/or adjust aspects of a current or future activity. In some embodiments, the developer platform 104 is configured as an Application Programming Interface (API) that includes a plurality of subroutines, each configured to perform one or more aspects of the methods set forth herein. It is contemplated, however, that the individual or combined functions of the plurality of subroutines may be implemented using other software or hardware configurations without departing from the scope of the disclosure.

As shown, the developer platform 104 includes one or more privacy-filter applications 134, a security module 136, a scoring module 138, an insights module 140, an analytics module 142, and a learning module 144. Beneficially, the developer platform 104 provides for an iterative reinforced learning process that may be used to provide signal analysis applications 106 with access to input data 120 received from peripheral devices 110 and non-platform software 112 in real-time while maintaining data privacy and security.

Typically the process begins when an analysis application 106 requests access to input data 120 from one or more peripheral devices 110 such as event streams received from a keyboard device or a mouse device, or a video stream received from a camera device. Each analysis application 106 may be configured to characterize one or more aspects of the user’s activities, health, wellbeing, or surroundings using data generated from one or more of the data sources 102 but typically does not require access to data generated by all available data sources. For example, an application configured to track a user’s posture may request access to video data from the user’s camera device but would likely not need or request access to keyboarding events from the user’s keyboard device. In some embodiments, access for each analysis application 106 is managed by the security module 136 based on permissions granted by the user, and if approved, the analysis application 106 may receive the requested data input stream in real-time via one of the one or more privacy-filter applications 134.

In some embodiments, one or more of the privacy-filter applications 134 include software algorithms configured to generate privacy-filtered event data 122a that is free of identifiable information, such as identifiable user information. The privacy-filtered data 122 may be generated by removing identifiable information from the input data 120, extracting non-identifiable data from the input data 120, and/or analyzing the input data 120 to generate non-identifiable data characterizing the input data 120, e.g., metadata based on the received input data. In some embodiments, the privacy-filtered data 122 are stored in memory (data storage 124) before and/or after use by an analysis application 106. As a security measure to maintain data privacy in the event of unauthorized access, the developer platform 104 typically does not store time-series input data received directly from a data source 102 (i.e., unfiltered data).

In one example, as illustrated in FIG. 3, an analysis application 106 (keyboarding analysis application 106a) may be configured to characterize aspects of the user’s keyboard use, such as duration of use, frequency of use, amount of use of certain keys (e.g., destructive or negative usage keys (e.g., backspace and delete keys) and constructive usage keys (i.e., non-negative or positive usage keys)), typing speed, and/or typing error rate. In this example, the keyboarding application 106a uses filtered event data 122a to perform the analysis where the filtered event data 122a is generated, using one or more event filters 134a, from input data 120 received from a keyboard device 114a, a mouse device 114b, and an operating system 112a. For example, the input data 120 may include key events 120a, mouse events 120b, and OS events 120c.

Here, the one or more event filters 134a are configured to generate privacy-filtered event data 122a free of undesired identifiable event information regarding a user. In some embodiments, the event filters 134a are configured to generate the filtered event data 122a through the use of standard event listeners, such as a keyboard event handling algorithm running in a loop that contains instructions to “listen for” or detect key events from a set of key events that do not provide enumeration information relating to the identity of individual character keys.

For example, depending on the programming language, a “KeyPress” event and a “KeyChar” event are typically generated when a user presses a character key on an ASCII keyboarding device, i.e., a key within the 128-character ASCII set of alphanumeric symbols. The KeyPress event identifies an activity generic to all character keys and the KeyChar event contains enumeration information for identifying a particular character key. To generate privacy-filtered event data, the keyboard event handling algorithm contains instructions to listen for the KeyPress event but does not contain instructions to listen for the KeyChar event. Thus, the algorithm can be used to detect when a character key has been pressed, but cannot be used to detect the identity of the character key. Typically, the algorithm contains instructions to detect both activity and enumeration key events for non-character keys. For example, the algorithm may be configured to detect “KeyUp” and “KeyDown” events used to identify activities generic to various directional keys (e.g., space, enter, tab, right arrow, left arrow), modifier keys (shift, alt, ctrl), and special keys (e.g., insert, delete, backspace), and key combinations for some keyboarding shortcut functions, such as cut and paste key combinations. The algorithm may also be configured to detect the specific enumeration events for non-character keys, so that the type of non-character key is captured in the filtered event data. Thus, the event filters 134a are configured to extract only non-character identifying information from each keystroke made on a keyboard. The extracted information will include a generic key event for printable characters, e.g., alphanumeric characters, such as key press or key release events, which do not include specific information relating to the key event that could be used to recreate the specific character keys pressed by the user. Thus, the event filters 134a are configured to ensure user data privacy by only generating non-identifiable data from the key events 120a.

In some embodiments, one or more of the directional key events, special key events (“destructive” key events), and the generic character key events (“constructive” key events) are used by the keyboarding application 106a to characterize aspects of keyboard use. For example, typing speed may be characterized using constructive key events and directional key events, e.g., “space” and “enter,” to infer words per minute (where spaces entered before and after one or more constructive key events indicate the typing of a single word). Similarly, typing accuracy may be characterized using a combination of constructive, directional, and destructive key events to infer the error rate per number of words typed (where directional and destructive key events may be used to infer the correction of an error). In some embodiments, mouse events combined with constructive or destructive key events may be used to approximate the deletion of blocks of text, such as mouse events related to right and left button clicks and movement.

It is contemplated that the manner of use of directional, destructive, and constructive keys may change based on the nature of the user’s task, e.g., coding, correspondence (email), word processing, and data analysis (spreadsheets). As a result, analysis results that might be viewed as positive for one type of task might be less favorable for a different type of task. Thus, in some embodiments, event data 122a provided by the operating system 112a, via the event filters 134a, may be used by the keyboarding application 106a to determine the nature of the task, e.g., by determining the active window for the corresponding key and mouse events, and adjust the respective analysis method, feedback scores and/or recommendations accordingly. In some embodiments, the keyboarding analysis application 106a is configured to periodically publish the analysis results, feedback scores, and/or recommendations to the developer platform 104 as a keyboarding analysis stream 126a.

In another example, as illustrated in FIG. 4, one or more of the signal analysis applications 106 may be configured to track a user’s posture (posture analysis application 106b), and one or more signal analysis applications 106 may be configured to track aspects of a user’s eye functions indicative of fatigue (e.g., the eye-fatigue analysis application 106c). Here, each analysis application 106b, 106c is configured to generate respective data analysis streams 126b, 126c using filtered video data 122b generated from a video signal 120d received from a camera device 114c. In some embodiments, the filtered video data 122b comprises metadata characterizing aspect of the image in the video signal 120d, where the metadata is generated using the video filter 134b. For example, in some embodiments, the video filter 134b includes one or more software algorithms configured to characterize user features within the video signal 120d, such as upper-body detection software and facial recognition software.

The upper-body detection software may be used to generate data characterizing non-identifiable aspects of the user’s upper body, such as the locations and orientations of various portions of the user’s upper body within the video. The facial recognition software may be used to generate data characterizing non-identifiable aspects of the user’s face, such as the position and orientation of various portions of the user’s face, the direction of the user’s gaze, whether the user is smiling or frowning, and the position of the user’s eyelids (used to determine eye-blink rate and/or squinting).

Here, the posture analysis application 106b may use the filtered video data 122b to determine aspects of the user’s posture and based thereon characterize one or more differences between the user’s posture and a desired ergonomic posture, e.g., leaning forward, leaning backward, elbows out, rounded shoulders, or asymmetric. Similarly, the eye-fatigue analysis application 106c may use the filtered video data 122b to generate analysis results related to eye strain and/or fatigue. For example, the eye-fatigue analysis application 106c may be configured to determine whether the user is squinting, the user’s eye-blink rate, and/or the amount of time since the user has looked away from the screen. Each of the signal analysis applications 106b, 106c may be configured to generate feedback, such as one or more feedback scores based on the analysis results, and/or one or more recommendations and periodically publish the results, feedback scores, and/or recommendations to the developer platform 104 as respective data analysis streams 126b, 126b.

In some embodiments, the input data 120 includes time-series data received by the developer platform 104 from a personal device 116, such as a wearable biometric device or a cell phone. In those embodiments, one or more of the privacy-filter applications 134 may remove identifiable information from the received input data 120 during a syncing operation or subsequent information transfer operations. In some embodiments, an application executed on the personal device 116 may remove identifiable information before transferring the input data 120 to the developer platform 104.

In some embodiments, one or more functions of the privacy-filter applications 134 and/or the analysis applications 106 may be performed by one or more non-platform software applications, such as one or more of the local non-platform software 112 executed on a user device, non-platform software executed on one of the interface devices 114, non-platform software 112 executed on a personal device 116, non-platform software executed on a sensor 118. In some embodiments, one or more functions of the privacy-filter applications 134 and/or the analysis applications 106 are performed using remotely executed non-platform software, e.g., by one or more virtual devices 152. In those embodiments, the non-platform software may function as a combination of a data source 102 and one or both of an analysis application 106 and/or a privacy-filter application 134.

In one example, as shown in FIG. 5, at least some of the signal stream information 126 is provided by one or more peripheral virtual devices 152, such as the gaming statistics tracker 152a and a music streaming service 152b. In this example, the gaming statistics tracker 152a and music streaming service 152b are executed remotely, but it should be noted that one or both may also be executed locally on a user device and/or may be executed using a personal device 116 in communication with the user device. Each of the gaming statistics tracker 152a and music streaming service 152b is configured to periodically publish information that may be used, based on permissions granted by the user, as signal stream information 126.

In this example, the gaming statistics tracker 152a may be configured to provide time-indexed data related to wins/losses, kills/deaths, total matches played, total time played, weapons used, highest kill games, longest win streaks, etc., each of which may be provided to the developer platform 104 as gaming statistics information 126d. The music streaming service 152b may be configured to provide time-indexed data, e.g., soundtrack information 126f, characterizing attributes of music provided to the user using any listening device, whether or not the listening device is in communication with the user device. The soundtrack information 126f provided by a music streaming service 152b such as Spotify® may characterize attributes such as song temp (beats per minute), song energy, danceability, loudness, valence (an indicator of positive mood for a song), duration, instrumentalness, acousticness, popularity, etc. Both the gaming statistics information 126d and the soundtrack information 126f may be periodically published to the developer platform 104 and presented to the user, e.g., by use of the user interface 108 without processing by a separate analysis application 106.

In some embodiments, signal stream information 126 generated by one or more signal analysis applications 106 or non-platform software 112, 152 may form a portion of input data 120 used by other signal analysis applications 106, e.g., to generate new signal stream information. For example, as shown in FIG. 5, a gaming analysis application 106s may be granted permission, by use of the security module 136, to receive privacy-filtered data 122 generated by the user’s interaction with a gaming application 112d and/or a gaming controller 114d as well as data contained in the signal analysis information, here the soundtrack information 126f and the gaming statistics 126d, provided by the music streaming service 152b and gaming statistics tracker 152a respectively. The gaming analysis application 106b may be configured to perform one or more calculations on the received data to generate gaming information 126e characterizing one or more relationships between a user’s gaming performance as provided in the gaming stats 126d, the user’s interactions with the gaming application 112d and or gaming controller 114e, and attributes of the music listened to during and/or proximate to the gaming activity as provided in the soundtrack information 126f.

In some embodiments, the developer platform 104 further includes a score module 138 configured to generate one or more composite feedback scores based on signal stream information 126 received from the plurality of signal analysis applications 106. The signal stream information 126, and the one or more composite feedback scores are included in the generated dashboard information 128, which is received from the developer platform 104 by the user interface 108 and represented to the user in a comprehensive dashboard 132.

In the example dashboard 132, illustrated in FIGS. 6-7, signal stream information 126 for each of the plurality of signal analysis applications 106 is represented in a timeline view, e.g., horizontally oriented graphs that share a common time axis, so that a user may better visualize the relationships therebetween. Factors or events affecting those relationships may be manually input by the user through the user interface 108 and/or generated using the insights module 140. In some embodiments, the insights module 140 includes one or more machine-learning artificial intelligence (Al) algorithms trained to generate system insights based on past, concurrent, or predicted changes or trends in the analysis results and/or based on relationships between signal stream information 126, e.g., data analysis streams 126a-d, generated using different ones of the plurality of signal analysis applications 106 and user insights 129.

User and system insights may be used to capture otherwise unmeasured factors or untracked events that indicate or affect an aspect of the user’s performance, health, wellbeing, or surroundings. Descriptors (tags) for user insights may be suggested by the user interface, e.g., via a dropdown menu, or may be determined by the user. Non-limiting examples of user insight tags include events, e.g., “consumed a cup of coffee” or “brisk walk,” the user’s mental and/or physical state, e.g., the user’s perceived performance, health, or wellbeing, such as “in the zone,” “focused,” “fatigued,” “stuck,” “distracted,” or ambient factors not captured from an existing data source, e.g., “my cat is on my lap.” In some embodiments, the developer platform 104 is configured to request, via the user interface 108, insights from the user, e.g., by periodically requesting wellbeing updates or by soliciting user insights based on the determined changes or trends in signal stream information 126, e.g., data analysis streams 126a-d, generated using one or more of the plurality of signal analysis applications 106.

In some embodiments, system insights 130 include events inferred from changes in signal stream information 126, determined from information received from one or more of the plurality of data sources 102, or both. For example, the system insight “away from the computer” might be inferred from a period of non-use of interface devices 114, such as a keyboard or a mouse, or may be determined based on an analysis of video data generated by a camera device that concludes the user was in fact, away from the computer. In some embodiments, the system insights are communicated to the user interface 108 and, with the user insights, are displayed on the dashboard 132 as time tags 131. In some embodiments, individual time tags 131 are presented as vertically oriented columns overlapping a stacked plurality of horizontally oriented data analysis stream charts, such as illustrated in FIGS. 6-7.

In some embodiments, the system 100 is configured for reinforced learning of user behavior, health, wellbeing, and ambient factors by use of one or a combination of the analytics module 142, the learning module 144, and the privacy quantization module 146, as illustrated in FIGS. 2-5. For example, the analytics module 142 may be configured to analyze the signal stream information 126 generated by one or more of the signal analysis applications 106, compare the signal stream information 126 generated by different signal analysis applications 106, compare the signal stream information 126 generated by one or more of the signal analysis applications 106 to user or system-generated insights 129, 130, or any combination thereof, and make a determination as to the quality or accuracy of the signal stream information 126 and/or a system-generated insight 130. The analytics module 142 may provide the determinations as feedback to respective signal analysis applications 106, which may use the feedback to change aspects of the methods used to determine the analysis results, scores, and/or recommendations contained in the signal stream information 126. Feedback, as determined by the analytics module 142, may also be provided to the insights module 140 for use in improving system-generated insights 130. Here, feedback generated by the analytics module 142 is analyzed using one or more of the privacy-filter applications 134 to remove potentially identifiable information before being provided to a signal analysis application 106 and/or stored in data storage 124.

The learning module 144 is configured to analyze information generated by the user (user insights) and one or more of the applications or modules of the system 100, e.g., the privacy-filter applications 134, analysis applications 106, the scoring module 138, the insights module 140, and the analytics module 142, and identify relationships in the data. In some embodiments, the learning module 144 is configured to determine factors affecting the identified relationships, provide feedback, and/or make a recommendation to the user to the user based on the identified relationships.

In one example, the learning module 144 is configured to identify relationships in the data based on proximate or concurrent changes or trends in signal stream information 126 generated using two or more signal analysis applications 106, such as a concurrent increase in eye-blink rate and typing errors, identify common factors affecting both, such as too much screen time without a break, and make a recommendation to the user, such as suggesting a walk or a cup of coffee. In another example, the learning module 144 is configured to identify relationships based on intersections or proximity of changes or trends in the signal stream information 126 for one or more of the signal analysis applications 106 with user insights 129 and/or system insights 130, such as a decrease in eye-blink rate and typing errors after a user insight of “got a cup of coffee” or a system insight of “away from the computer.”

In some embodiments, the learning module 144 is a machine learning Al algorithm trained to identify relationships between data. In some embodiments, the learning module 144 is configured to train the Al algorithm using information generated by the system 100. Thus, based on an analysis performed by the machine learning Al algorithm, user specific or tailored insights can be created that can be displayed on the dashboard 132 or transmitted to the user via an acceptable method.

In some embodiments, the developer platform 104 further includes a privacy quantization module 146 that may be used to analyze relatively large amounts of data and produce a transformed data set containing anonymized information (free of potentially identifiable information) in a simplified format suitable for processing by the one or more privacy-filter applications 134 and/or storage in memory (data storage 124). For example, in some embodiments, the privacy quantization module 146 is configured to perform one or more signal processing operations to convert relatively large volumes of continuously received input data 120, e.g., a video stream or audio stream, into a smaller manageable data sets, e.g., quantized data 150, suitable for storage in memory of a user device. In some embodiments, the privacy quantization module 146 is configured to identify potentially privacy-sensitive information within the input data 120, e.g., facial features that may be used to determine the user’s identity, user’s health related information or demographic information, and perform one or more privacy-preserving data processing operations to remove or obscure the privacy-sensitive information, such as intentionally introducing noise during a compression operation to convert input data 120 into quantized data 150. In some embodiments, the privacy quantization module 146 is used to process input data 120 before and/or after processing by the privacy-filter applications 134 so that the privacy-filtered data 122 comprises quantized data 150. In some embodiments, the privacy quantization module 146 is configured to generate quantized data 150 from signal stream information received from the analytics module 142. Generally, access to and storage of the quantized data 150 is controlled by the security module 136.

### EXAMPLE DASHBOARD

FIGS. 6-7 depict different views of an example dashboard 132 generated for display to a user by the user interface 108, according to one embodiment. Here, the dashboard 132 includes a timeline section 602, a detail section 604, and a control section 606, where each section is interactive so that a user can select between different views by use of a graphical user interface, such as between the different views shown in FIGS. 5 and 6, respectively.

As illustrated in FIGS. 6-7, the timeline section 602 includes a plurality of signal stream charts 608 (e.g., charts 608a-608e) visually represented in an opaque background and a plurality of time tags 131 visually represented as semi-transparent columns 610 that overlay the opaque signal stream charts 608. Each of the plurality of signal stream charts 608 graphical represents time-series results data received in signal stream information 126, e.g., one of the plurality of data analysis streams 126a-d, and each of the time tags 131 provides a visual representation of a time-indexed user or system insight 129, 130. Beneficially, the plurality of signal stream charts 608 and the plurality of columns 610 are disposed in an arrangement that enables a user to visualize the temporal relationships therebetween, and thereby be able to understand the relationships between the information provided in the signal stream information 126 and time tags 131 so that the user can, for example, better understand their behavior, aspects of their health, aspects of their wellbeing, and/or aspects of how their surroundings are affecting them.

In the example shown the plurality of signal stream charts 608 include an ambient light chart 608a, an eye fatigue chart 608b, a calendar chart 608c, a soundtrack chart 608d, and a keyboard chart 608e, which are arranged in a vertical stack so that each one of the plurality of signal stream charts 608 is adjacently above or below another one of the signal stream charts 608a-e. The signal stream charts 608a-e share the time axis 612 so that the time-time series results data represented in each of the signal stream charts 608a-e is temporally aligned in the vertical direction.

The plurality of time tags 131 each correspond to a time-indexed user or system insight represented as vertically oriented semi-transparent columns 610 temporally aligned on the time axis 612 with the signal stream charts 608a-e. Each of the semi-transparent columns 610 is overlaid across the collective plurality of signal stream charts 608a-e to intersect the time-series results data represented therein. For some of the time tags 131, a width W of the semi-transparent column 610 corresponds to a discrete time period, e.g., 15 minutes for “away from the computer.” For some time tags 131, the semi-transparent column 610 is relatively narrow, e.g., a vertical line marking the time of a user insight, e.g., “feeling in a fog.” In some embodiments, the semi-transparent columns 610 are color-coded depending on the information contained in the time tag 131. For example, time tags 131 where the user is away from the user device may be represented in a semi-opaque blue color, tags with a negative association, such as “feeling fatigued,” may be represented in a semi-opaque orange color, and tags with a positive association, such as “in the zone,” may be represented in a semi-opaque green color.

In other embodiments, user and system generated insights 129, 130 may be presented in one or more horizontally oriented insight charts (not shown). In those embodiments, the insight charts may be arranged in the vertical stack with the signal stream charts 608k, e.g., adjacently above or below one or more of the signal stream charts 608 and share the common time axis 612 therewith. In some embodiments, textual information contained in the signal stream information 126 is presented to the user in a signal stream message section 624.

In some aspects of the disclosure, the timeline section 602 is configured so that the user may select between a first view 602a (FIG. 6) and a second view 602b (FIG. 7). In each view, the timeline section 602 is configured to represent a calendar day, i.e., from 12:00AM to 11:59 PM. In the first view 602a, time is represented using a linear scale so that tick marks 614 representing equal time increments, e.g., 10-minute increments, are equidistant from one another along the axis 612, so each 10-minute increment is represented by a segment of the axis 612 that spans a distance S. The first view 602a allows the user to visualize trends in the represented data so that the user can track desired signal stream information 126 during the course of the day. In some embodiments, the first view 602a includes a plurality of feedback scores 616, each corresponding to one of the plurality of signal stream charts 608 and displayed adjacent thereto. Typically, each of the plurality of feedback scores 616 are included in the signal stream information 126 generated by the signal analysis applications 106.

The second view 602b (FIG. 7) allows the user to explore desired portions of the signal stream charts 608 in more detail by horizontally distorting the axis 612 to expand the view for a time period 618 selected by the user. The expanded view for time period 618 is provided by stretching first distances S₁ between tick marks 614 within the time period 618 and compressing second distances S₂₋ₙ between tick marks 614 on each side of the time period 618.

The detail section 604 has at least a momentary detail view 604a, as shown in FIG. 6, and a daily detail view 604b (FIG. 7). The momentary detail view 604a provides the user with information generated by a signal analysis application 106 that may or may not otherwise be available in the corresponding signal stream chart 608. In some embodiments, the momentary detail view 604a includes information generated by the signal analysis application 106 that characterizes aspects of the user’s behavior, health, wellbeing, and/or surroundings that are related to but not displayed in the corresponding signal stream chart 608. In some embodiments, the momentary detail view 604a includes one or more recommended actions 617 generated by the signal analysis application 106 that the user may take to improve the corresponding feedback score 616. In the example shown, the momentary detail view 604a displays the individual aspects of the user’s upper body position used to characterize the user’s posture, a written summary of the user’s posture, “leaning too far forward,” and a recommended action that the user can take to improve their posture, “pull your shoulders back and straighten your spine.” The daily detail view 604b (FIG. 7) provides the user with a written summary of a system-generated analysis of the information contained in the signal stream information 126 and recommended actions 619 the user can take to improve an aspect of their performance, health, or wellbeing. In some embodiments, the recommended actions may be used to improve an overall feedback score (not shown).

The control section 606 contains one or more interactive features that allows the user to configure and/or customize the dashboard 132, such as by use of the slider bar 622 to adjust the number of signal stream charts 608 displayed in the timeline section 602 or by use of the time tag emoji 620 to enter predetermined time tags of feeling in the zone (flexed arm emoji) or feeling in a fog (neutral face emoji). In some embodiments, the control section 606 is used to display an overall feedback score (not shown) generated by the score module 138 based on an analysis of the information contained in signal stream information 126 generated using more than one of the signal analysis applications 126.

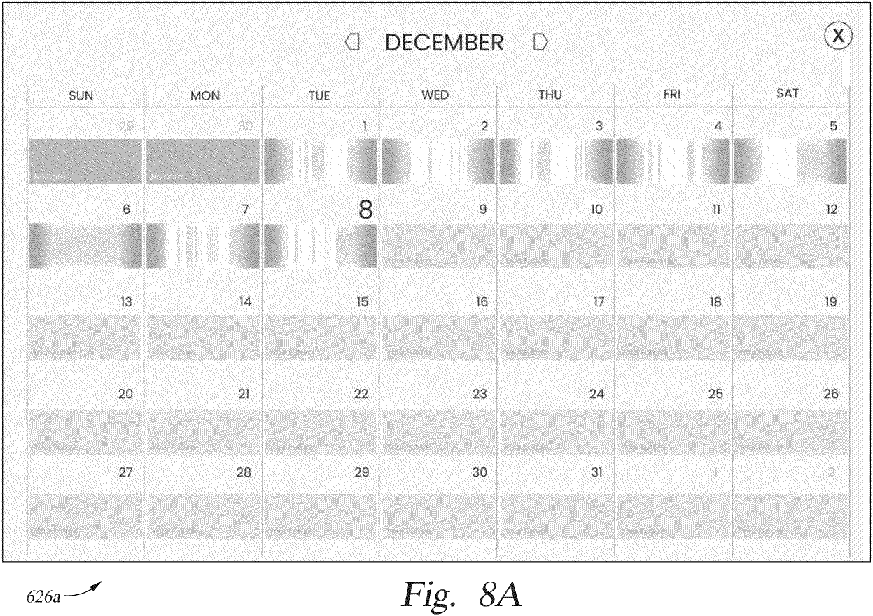

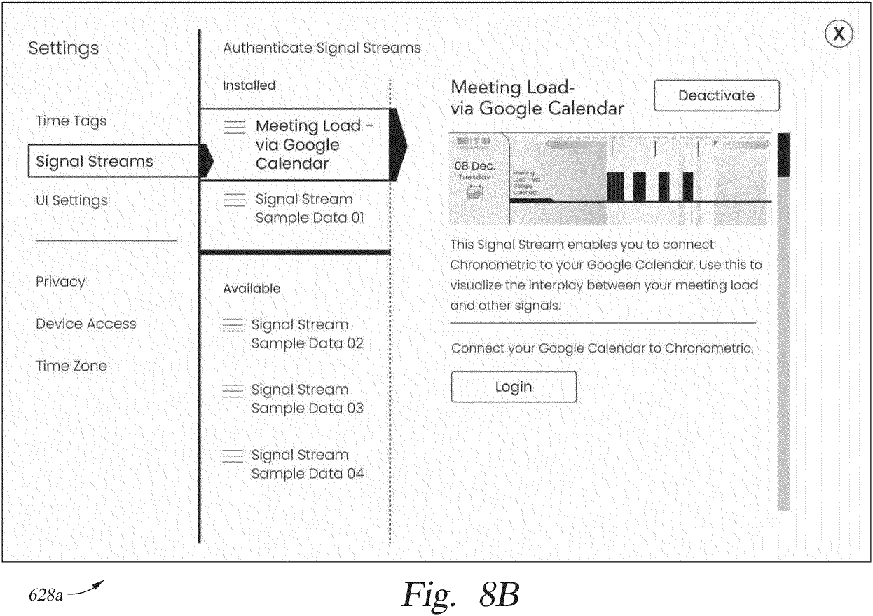

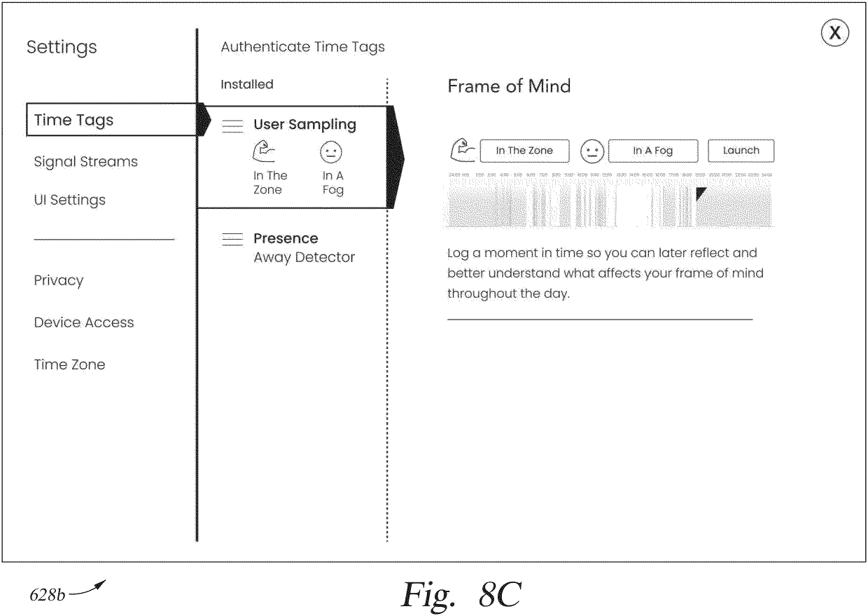

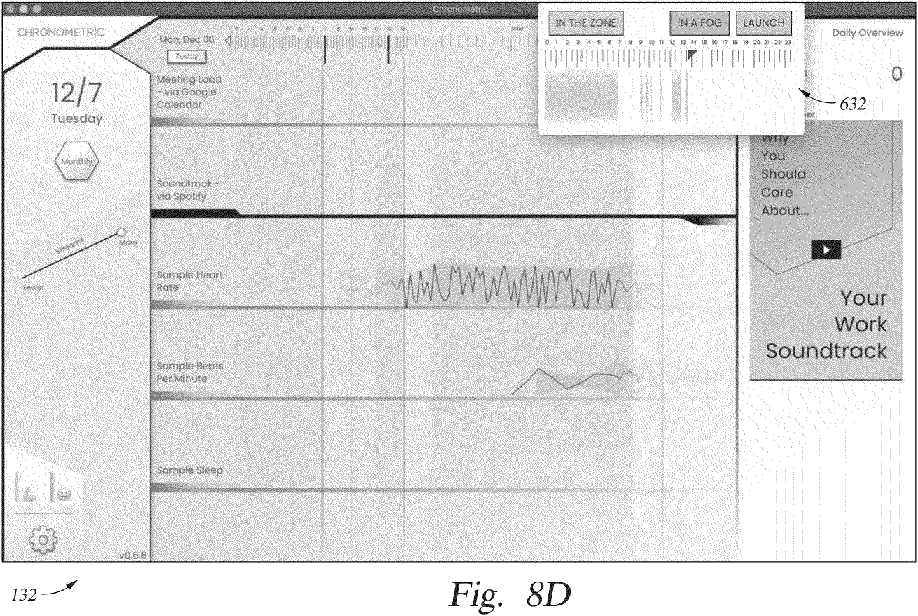

Here, the control section 606 further includes a monthly view button 626 to display the monthly view 626a shown in FIG. 8A and a settings button 628 to take the user to the settings menu 628a discussed in relation to FIGS. 8B-8C. As shown in FIG. 8A the monthly view 626a provides the user with a visual display of one or more time tags, shown here as the “away from the computer” time tag, although other time tags can be selected. The settings menu 628a (FIGS. 8B-8C) allows the user to select desired signal stream information 126 for display to the user as signal stream charts 608 (FIG. 8B), configure predetermined time tags and/or enter custom time tags (FIG. 8C), as well as control privacy and security aspects, e.g., privacy-filtering settings for input data 120 and access to privacy-filtered data by third-party applications. Here, the settings menu 628a is also configured to enable a user to temporally align input data 120, signal stream information 126, and insights 129, 130 when traveling across different time zones.

FIG. 8D is a screen shot of a glance view 632 of the user interface 108. Here the glance view 632 is a simplified view of the dashboard 132 depicting time tags across the same 24 hour period. Typically, the glance view 632 is configurable to run as a background display on the user’s operating system interface and/or overlay other applications.

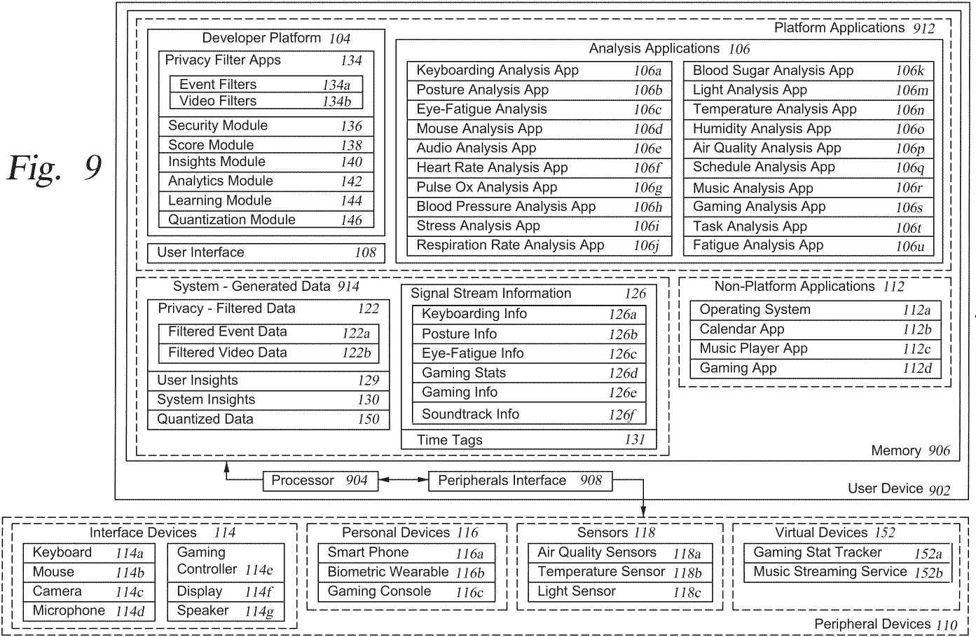

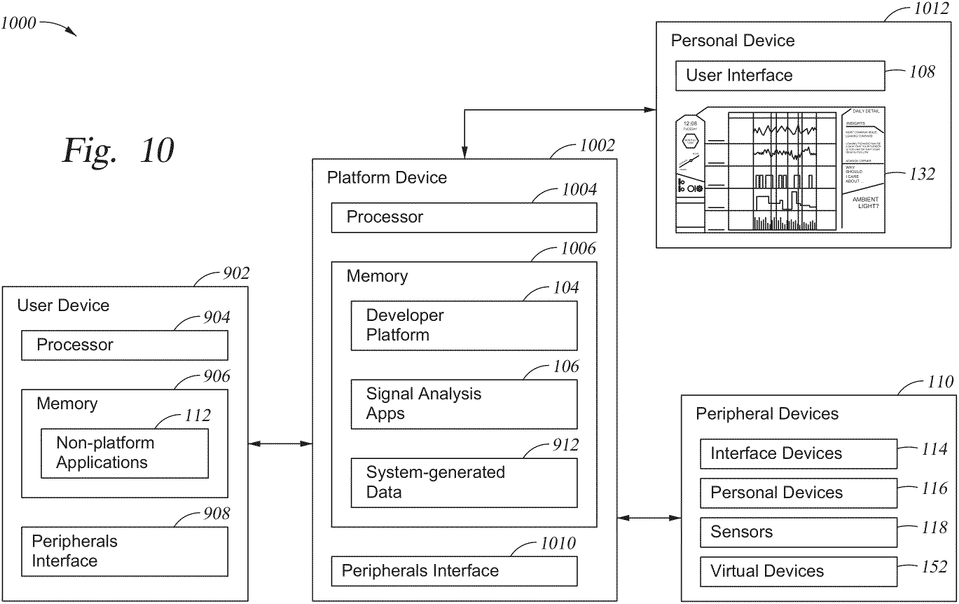

In some embodiments, the user interface 108 used to generate and display the dashboard 132 is executed on the same device as the developer platform 104 and the signal analysis applications 106, such as the user device 902 illustrated in FIG. 9. In other embodiments, one or more of the developer platform 104, the signal analysis applications 106, and the user interface 108 are executed on one or more devices peripheral to the user device 902, such as described in relation to FIG. 10.

### EXAMPLE SYSTEM PLATFORMS

FIG. 9 is a block diagram of an example user device 902 configured to implement the systems and methods described herein, according to one embodiment. Here, the user device 902 is a personal computing device, e.g., a desktop or laptop computer, configured with hardware and software that may be employed by a user to engage in routine computer-related activities, such as computer-related work or gaming activities. As shown, the user device 902 includes a processor 904, memory 906, and a peripherals interface 908. The processor 904 may be any one or combination of a programmable central processing unit (CPU), a graphics processing unit (GPU), a digital signal processor (DSP), a video signal processor (VSP) that is a specialized DSP used for video processing, a field programmable gate array (FPGA), an application-specific integrated circuit (ASIC), a neural network coprocessor, or other hardware implementation(s) suitable for performing the methods set forth herein, or portions thereof.

The memory 906, coupled to the processor 904, is non-transitory and represents any non-volatile memory of a size suitable for storing one or more non-platform software 112, one or more platform applications 912, and system-generated data 914 as described below. Examples of suitable memory that may be used as the memory 906 include readily available memory devices, such as random access memory (RAM), flash memory, a hard disk, or a combination of different hardware devices configured to store data. In some embodiments, memory 906 includes memory devices external to the user device 902 and in communication therewith.

Here, the one or more platform applications 912 include the subroutines of the developer platform 104, the plurality of signal analysis applications 106, and the user interface 108, each of which is stored in memory 906 and includes instructions that, when executed by the processor 904 are configured to perform respective portions of the methods described herein. The individual subroutines of the developer platform 104 and example signal analysis applications 106a-u shown in FIG. 9 are described elsewhere in this disclosure and are therefore not recited again here.

The peripherals interface 908 is configured to facilitate the transfer of data between the user device 902 and one or more of the plurality of peripheral devices 110, including input/output (“I/O”) devices, here interface devices 114, that are integrated with or are disposed in wired or wireless communication with the user device 902, personal devices 116 that are independently operable to generate and store input data 120, and sensors 118 configured to measure ambient conditions in the user’s environment. The peripherals interface 908 may include one or more USB controllers and/or may be configured to facilitate one or more wireless communication protocols that may be used may include, but are not limited to Bluetooth, Bluetooth low energy (BLE), Infrastructure Wireless Fidelity (Wi-Fi), Soft Access Point (AP), WiFi-Direct, Address Resolution Protocol (ARP), ANT UWB, ZigBee, Wireless USB, or other useful personal area network (PAN), wide area network (WAN), local area network (LAN), wireless sensor network (WSN/WSAN), near field communication (NFC) or cellular network communication protocols.

FIG. 10 is a block diagram of a system 1000, according to one embodiment, which is configured to execute one or more of the platform applications 910 on a device other than the user device 902. It is contemplated that the system 1000 may be used in circumstances where it is not desirable or feasible to install the platform applications 910 on a device predominantly used by the user to accomplish routine computer-related tasks, such as an employer owned computer or some types of gaming consoles.

As shown, the system 1000 includes the user device 902, configured as described in relation to FIG. 9, and a platform device 1002 disposed in wired or wireless communication with the user device 902. The platform device 1002 includes a processor 1004, memory 1006, and a peripherals interface 1010 each of which may be configured the same or similarly to the respective processor 904, memory 906, and peripherals interface 908 described above for the user device 902. The system 1000 further includes a personal device 1012, e.g., a smartphone or a tablet, in communication with the platform device 1002. Here, the platform device 1002 is configured to execute, by use of the processor 1004, the various subroutines of the developer platform 104 and the plurality of signal analysis applications 106 which are stored in memory 1006. The personal device 1012 is configured to execute the user interface 108 and display the interactive dashboard 132.

As shown, the system 1000 is configured so that the platform device 1002 receives input data 120 directly from the plurality of peripheral devices 110 and from the non-platform software 112 directly from the user device 902 through wired or wireless communication with each that is facilitated by the peripherals interface 1010. The user device 902 may receive information from one or more of the interface devices 114 through the platform device 1002 and/or the interface devices 114 may communicate with both the user device 902 and the platform device 1002 directly. In some embodiments, the platform device 1002 is integrated with an interface device 114, e.g., the keyboard device 114a, mouse device 114b, camera device 114c, microphone 114d, and/or gaming controller 114e.

### EXAMPLE METHODS

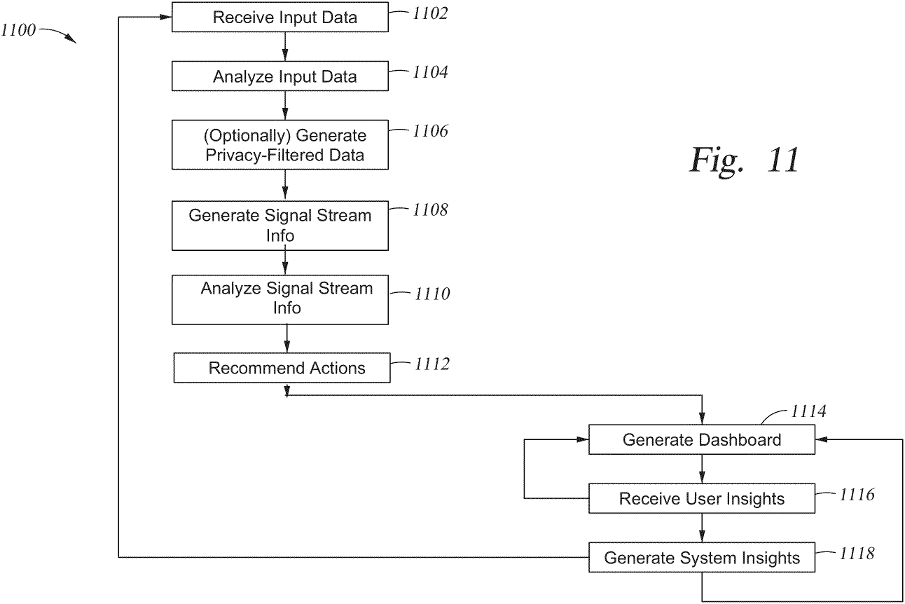

FIG. 11 is a flow diagram illustrating a method that may be performed using the systems described herein, according to one embodiment. The method 1100 begins at block 1102 with receiving input data 120 from a plurality of peripheral devices 110. In some embodiments, the input data 120 is generated from a user’s interaction with one or more interface devices 114. In some embodiments, input data 120 includes event data, such as key events from a keyboard device1 14a, motion and click events from a mouse device 114b, and/or motion and button events from a gaming controller 114e. In some embodiments, input data 120 includes video signals from a camera device 114c and/or audio signals from a microphone 114d. In some embodiments, input data 120 includes signals sent to or received from output devices, such as a display device 114f and/or one or more speaker devices 114g. Generally, input and output signals from the interface devices 114 are received by the peripheral interface 908 and processed in real-time to provide time-series input data 120 to the developer platform 104.

In some embodiments, input data 120 are received from one or more personal devices 116, such as a smartphone 116a, one or more personal biometric devices 116b, or other personal devices, such as medical devices, activity trackers, and location trackers. Input data 120 from personal devices 116 may be received in real-time as described above or may comprise packets of time-series data received at the user device 902 during periodic syncing operations.

In some embodiments, input data 120 are received from sensors 118 used to monitor ambient conditions in the user’s environment, such as air quality sensors 118a, temperature sensors 118b, and light sensors 118c. In some embodiments, one or more of the sensors 118 may be integrated with another peripheral device, such as an ambient light sensor used to adjust the brightness of the display device 114f. In some embodiments, input data 120 are received from one or more non-platform software 112 executed on the user device 902, such as the operating system 112a, calendaring applications 112b, music player applications 112c, gaming applications 112d, or other non-platform software. In some embodiments, one or more of the non-platform software 112 are executed on a platform device 1002, such as described in relation to FIG. 10, and input data 120 are received therefrom.

At block 1104, the method 1100 includes analyzing input data 120 to generate a plurality of data analysis streams. Here, analyzing input data 120 to generate a plurality of data analysis streams optionally includes generating privacy-filtered data 122 at block 1106 and generating signal stream information at block 1108.

At block 1106, the method 1100 includes receiving the input data 120 at the developer platform 104 and (optionally) filtering the input data 120 by use of one or more privacy-filter applications 134 to generate privacy-filtered data 122 that is free of identifiable and/or sensitive user information. Generating the privacy-filtered data 122 may include remove identifiable information from the input data 120, extracting non-identifiable information from the input data 120, analyzing the input data 120 to generate non-identifiable data that characterizes the input data 120, i.e., metadata, or a combination thereof. In some embodiments, generating privacy-filtered data 122 includes generating filtered event data 122a for use by the keyboarding analysis application 106a, such as described in relation to FIG. 3. In some embodiments, generating privacy-filtered data 122 includes generating filtered video data 122b for use by the posture analysis application 106b and the eye-fatigue analysis application 122c, as described in relation to FIG. 4.

At block 1108, the method 1100 includes generating, by use of a plurality of signal analysis applications 106, signal analysis information 126 comprising a plurality of data analysis streams. In some embodiments, one or more of the signal analysis applications 106 are third-party applications configured to interface with the developer platform 104 by use of a software developer kit. Each of the respective signal analysis applications 106 are configured to perform one or more calculations on the input data 120 or privacy-filtered data 122 to characterize one or more aspects of a user’s activities, health, wellbeing, behavior, or surroundings. Thus, each of the respective signal analysis applications 106 may utilize input data from one or a plurality of data sources 102 to generate one or more data analysis streams.

In some embodiments, one or more of the signal analysis applications 106 are configured to characterize a relationship between at least two aspects of the user’s activities, behavior, health, wellbeing, or surroundings. In some embodiments, one or more of the signal analysis applications 106 are configured to compare the analysis results to desired results and generate a feedback score that may be used to gauge and track improvements in user behavior, health, wellbeing, or surrounding conditions over time. In some embodiments, one or more of the signal analysis applications 106 are configured to generate recommended actions that the user can take to improve the analysis results and/or feedback score.

The signal analysis applications 106a-u described below provide nonlimiting examples of applications that may be used to a generate signal stream information 126 comprising a plurality of data analysis streams using data received from interface devices 114, personal devices 116, sensors 118, non-platform software 112, or combinations thereof. Examples of analysis applications configured to generate data analysis streams based on data received from interface devices 114 include the keyboarding analysis application 106a (described in relation to FIG. 3), a posture analysis application 106b and an eye-fatigue analysis application 106c (each described in relation to FIG. 4), a mouse movement analysis application 106d, and an audio analysis application 106e (e.g., microphone analysis).

Examples of signal analysis applications 106 configured to generate signal stream information 126 using data received from personal devices 116, such as biometric devices 116b and activity trackers, include a heart rate analysis application 106f, oxygen saturation and pulse rate analysis app, (e.g., pulse ox analysis application 106g), a blood pressure analysis application 106h, a stress analysis application 106i (e.g., galvanic skin response), a respiration rate analyses application 106j, and a blood sugar analysis application 106k.

Examples of analysis applications configured to generate signal analysis data based on data received from sensors 118 include an ambient light analysis application 106m, a temperature analysis application 106n, a humidity analysis application 106o, and an air quality analysis application 106p. In some embodiment the air quality analysis application 106p is a CO₂ level analysis application. Examples of analysis applications configured to generate signal analysis data based on data received from non-platform software 112 include a schedule analysis application 106q to analyze data from a calendaring application 112b, a music analysis application 106r to analyze data from a music player application 112c, a gaming analysis application 106s to analyze data from a gaming application 112d, and a task analysis application 106t to analyze data received from the operating system 112a.

In some embodiments, one or more of the signal analysis applications 106 are configured to generate signal analysis data that characterize a relationship between at least two aspects of the user’s activities, behavior, health, wellbeing, and surroundings. In one example, a task analysis application 106t may be configured to generate a data analysis stream characterizing one or more of the data analysis stream results described above for a particular computer-related task determined from OS event data, e.g., typing error rate while coding or posture while reading emails. In another example, a fatigue analysis application 106u may be configured to generate a data analysis stream characterizing a relationship between two or more indicators of fatigue, such as typing error rate or eye-blink rate, or between one more indicators of fatigue and one or more factors affecting fatigue, such as blood sugar levels, CO₂ levels, meeting load, or time at the user device.

In some embodiments, one or more of the example analysis applications 106a-u are configured to generate a data analysis stream 126 with multiple characterizations within a category described by the signal analysis application 106a-u. For example, the posture analysis application 106b may generate a data analysis stream 126b that characterizes multiple aspects of the user’s posture including whether the user was leaning forward, leaning backward, had their elbows out, had rounded shoulders, or was leaning to one side (asymmetric). So that the user in not inundated with posture related timelines in the dashboard 132, the posture analysis application 106b may generate a posture feedback score based on an analysis of two or more of the posture characterizations. The posture score may be displayed as a posture timeline so that the user can see posture related trends or changes and the individual posture related characterizations may be represented in the momentary detail view, as shown in FIG. 6. In some embodiments, one or more of the example analysis applications 106a-u described above may correspond to a category having a plurality of signal analysis applications, each configured to generate a corresponding data analysis stream 126.

In one example, a first data analysis stream of the plurality of data analysis streams is generated using a first input signal from an interface device, such as a keyboard device, and a second data analysis stream of the plurality of data analysis streams is generated using a second input signal received from a biometric sensor. In this example, the first data analysis stream characterizes one or more aspects of the user’s interactions with a user device and the second data analysis stream characterizes one or more aspects of the user’s physical activity, health, or wellbeing. In another example, an additional third data analysis stream of the plurality of data analysis streams is generated using a third input signal received from a sensor configured to measure one or more ambient conditions, and the third data analysis stream characterizes one or more ambient conditions experienced by the user.

One or more of the signal analysis applications 106 may be configured to generate corresponding signal stream information 126 using input data 120 received at the developer platform 104 and processed by one or more of the privacy-filter applications 134 in real-time. In some embodiments, the privacy-filtered data 122 is concurrently received and analyzed by the signal analysis application 106 to generate analysis results, which are periodically published to the developer platform 104 along with the feedback scores and recommended actions, such as at intervals between about 30 seconds and 5 minutes. Other ones of the signal analysis applications 106 may be configured to generate a data analysis stream 126 using input data 120 received at the developer platform 104 in batches, such as through a syncing operation with the data source 102. Typically, the input data 120 received through a syncing operation is times-series data which may be filtered using a privacy-filter application 134, analyzed using a signal analysis application 106, and published in batches to the developer platform 104 as time-series data within the data analysis stream 126.

At block 1110, the method 1100 includes receiving the plurality of data analysis streams at the developer platform 104, and analyzing two or more of the data analysis streams to generate feedback that may be implemented by the user to improve their performance health or wellbeing, such as an (optional) overall feedback score. In one example, the a music player application 112c is configured to provide signal stream information relating to an audio signal that is being provided to a user (e.g., information can include audio playback sound level, song type, beats per minute, etc.) and the keyboarding analysis application 106a is configured to provide one or more data analysis streams containing keyboarding information 126a relating to a user’s mouse activity (e.g., mouse movement speed) or keyboard activity (e.g., typing speed). In this example, the analysis at block 1108 may be used to determine that certain songs or audio related environmental factors can have a positive or negative effect on the user’s ability to perform certain tasks and thus allow an overall feedback score to be generated that is commensurate to the positive or negative effect one data analysis stream has on the other.

At block 1112, the method 1100 includes generating one or more recommended actions based on the analysis at block 1108. Here, the signal stream information is received and analyzed by a score module 138, which, based on the analysis, generates one or more recommended actions 619 that the user can take to improve the overall feedback score. The score module 138 periodically publishes dashboard information 128 comprising the signal stream information 126, e.g., the plurality of data analysis streams, the overall feedback score, and the recommended actions 619 to the user interface 108 for display to the user by use of the dashboard 132.

At block 1114, the method 1100 includes receiving the dashboard information 128, user insights 129, and system insights 130 at the user interface 108 and generating a dashboard 132 for display to the user. User insights 129 and system insights 130 are respectively determined in blocks 1116 and 1118 of the method 1100 as described below. Generally, the dashboard 132 is configured to display a plurality of signal stream charts 608 and a plurality of time-tag representations (semitransparent columns 610), such as shown in the example dashboard 132 of FIGS. 6-7. The plurality of signal stream charts 608 are aligned by a common time axis 612 and each chart 608 graphically represents time-series data received in the signal stream information 126, e.g., one of the plurality of data analysis streams over a first period of time. The plurality of time-tag representations (e.g., columns 610 in FIGS. 6-7) represent user insights 129 and/or system insights 130 at second periods of time within the first period of time and may be displayed as vertically oriented columns 610 or lines that extend across the vertically stacked signal stream charts 608.

At block 1116, the method 1100 includes receiving user insights 129 at the user interface 108 and displaying the user insights 129 on the dashboard 132 as one or more of the time-tag representations. Typically, user insights 129 describe one or more events, ambient conditions, mental states, and/or physical states experienced by the user at respective second times or a second periods of time within the first period of time represented in the plurality of signal stream charts 608. The descriptors are used to “tag” the event, ambient condition, mental state, and/or physical state to the second periods of time and are referred to herein as “time tags.” The descriptors may be generated by the user or selected from a list of predetermined descriptors. The user insights 129 may be input by the user without prompting by the user interface 108, may be requested from the user as periodic wellbeing updates, and/or may be requested from the user based on determined changes or trends in the data analysis streams generated using one or more of the signal analysis applications 106. Once received, the user insights 129 may be displayed on the dashboard 132 as the time-tag representations described in block 1114. Typically, the user insights 129 are published to developer platform 104 for further analysis, e.g., by use of the insights module 140.

In some embodiments, user insights 129 are entered using one or more dedicated features of a peripheral device 110. For example, in some embodiments one of more interface devices 114, such as a keyboard device 114a or gaming controller 114e may be configured with dedicated entry keys, e.g., dedicated time tag keys that may be used to enter predetermined insights. In some embodiments, such dedicated time tag keys may have a visual representation of the time tag, such as a commensurate emoji for “in the zone” or “in a fog.”

At block 1118, the method 1100 includes generating system insights 130 and displaying the system insights 130 on the dashboard 132 as one or more of the time-tag representations. In some embodiments, generating system insights 130 includes determining that there are changes in at least two of the data streams that happened concurrently or proximately in time and, based on the determined changes, determining that an event has occurred. In some embodiments, generating the system insights 130 includes analyzing the plurality of signal stream information 126, e.g., one or more of the individual data analysis streams, and/or user insights 129 using a machine-learning artificial intelligence (AI) algorithm trained to infer events from changes within one or more of the data analysis streams, predict user performance based on learned user behaviors, and/or correlate analysis results to input data not otherwise tracked. Here, the system insights 130 are published to the user interface 108 for display on the dashboard 132 as one or more of the time tag representations.

The methods, systems, and devices described herein collectively provide a system platform that may be used beneficially to improve the effective use of time, performance of activities, health, and wellbeing of an individual user while maintaining the user’s data privacy and security.

While the foregoing is directed to embodiments of the present disclosure, other and further embodiments of the disclosure may be devised without departing from the basic scope thereof, and the scope thereof is determined by the claims that follow.

What is claimed is:

1. A computer-implemented method for improving user performance, health, and wellbeing, comprising:
a) receiving input data from a plurality of peripheral devices, the plurality of peripheral devices comprising one or more interface devices that are integrated with or in communication with a user device;
(b) analyzing the input data to generate signal stream information comprising a plurality of data analysis streams, each of the plurality of data analysis streams comprising time-series results data for a first period of time relating to a user;
(c) generating a plurality of time tags corresponding to second periods of time within the first period of time, wherein one or more of the plurality of time tags are based on insights relating to the user; and
(d) generating a dashboard for display, the dashboard comprising:
a plurality of data analysis stream charts aligned by a common time axis, each chart graphically representing the time-series results data over the first period of time for a respective one of the plurality of data analysis streams; and
a plurality of time-tag representations extending across the plurality of data analysis stream charts at the second periods of time.

2. The computer-implemented method of claim 1, wherein the time-series results data relating to the user comprises one or more aspects of the user’s performance of an activity, user’s health, user’s wellbeing, user’s behavior, or user’s surroundings.

3. The computer-implemented method of claim 1, further comprising:
(e) generating a first feedback score for display to the user based on data in at least two of the plurality of data analysis streams;
(f) generating one or more recommended actions based on information found in one of the at least two of the plurality of data analysis streams; and
(g) presenting the first feedback score and the recommended actions in the dashboard.

4. The computer-implemented method of claim 1, wherein the insights related to the user are generated by:
(i) determining that there are changes in at least two of the data analysis streams that happened concurrently or proximately in time; and
(ii) based on (i), determining that an event has occurred.

5. The computer-implemented method of claim 1, wherein the insights related to the user comprise information relating to the user’s mental or physical state.

6. The computer-implemented method of claim 1, wherein one or more of the insights relating to the user are based on an event experienced by the user.

7. The computer-implemented method of claim 1, wherein
a first data analysis stream of the plurality of data analysis streams is generated using a first input signal from a first device, and
the first data analysis stream characterizes one or more aspects of the user’s interactions with the user device.

8. The computer-implemented method of claim 7, wherein
a second data analysis stream of the plurality of data analysis streams is generated using a second input signal received from a second device, the second device comprising a biometric sensor, and the second data analysis stream characterizes one or more aspects of the user’s physical activity, health, or wellbeing.

9. The computer-implemented method of claim 8, wherein
a third data analysis stream of the plurality of data analysis streams is generated using a third input signal received from a third device,
the third device comprises a sensor configured to measure one or more ambient conditions, and
the third data analysis stream characterizes one or more ambient conditions experienced by the user.

10. The computer-implemented method of claim 7, wherein the first device is a keyboard device, and the first data analysis stream characterizes one or more aspects of the user’s interactions with the keyboard device.

11. The computer-implemented method of claim 10, wherein input data used to generate the first data analysis stream is privacy-filtered event data generated from the first input signal, the privacy-filtered event data comprising destructive key events and constructive key events, the destructive key events comprising delete or backspace key events and the constructive key events comprising one or more generic key events for printable characters.

12. The computer-implemented method of claim 11, wherein the privacy-filtered event data is free of key events that could be used to identify individual printable characters input by the user.

13. The computer-implemented method of claim 12, wherein analyzing the input data to generate the first data analysis stream comprises comparing respective counts of constructive key events and destructive key events over repeating intervals of time to periodically characterize one or both of the user’s keyboarding accuracy or keyboarding speed.

14. The computer-implemented method of claim 1, wherein the insights relating to the user are generated by:
(i) periodically requesting the user to select a user insight from a list of predetermined user insights; or
(ii) determining that there are changes in at least two of the data analysis streams that happened concurrently or proximately in time; and
(iii) based on (ii), requesting that the user select the user insight from the list of predetermined user insights or manually enter a description for a new user insight.

15. The computer-implemented method of claim 1, wherein analyzing the input data comprises generating privacy-filtered input data by:
(i) removing identifiable data from input data received from one or more of the plurality of peripheral devices;
(ii) extracting non-identifiable data from input data received from one or more of the plurality of peripheral devices; or
(iii) analyzing input data received from one or more of the plurality of peripheral devices to generate non-identifiable metadata.

16. The computer-implemented method of claim 1, wherein the one or more interface devices comprise a keyboard, a camera, a mouse, a microphone, or a gaming controller.

17. A computer-implemented method for improving the performance of one or more user activities, comprising:
(a) receiving, by a user device, time-series input data generated from a user’s interaction with one or more interface devices that are in communication with the used device;
(b) analyzing the time-series input data to generate signal stream information comprising a plurality of data analysis streams, each of the data analysis streams containing time-series results data formed within a first period of time;
(c) receiving, by use of a user interface application, user insights describing one or more events, ambient conditions, behaviors, mental states, and/or physical states experienced by the user at one or more second periods of time within the first period of time;
(d) generating one or more system insights, comprising:
(i) determining that an event has occurred by determining that there are changes in at least two of the data analysis streams that happened concurrently or proximately in time; or
(ii) determining a relationship between one or more of the data analysis streams and a user insight by identifying one or more factors that affect the relationship, wherein the one or more factors are identified by comparing one or more rules stored in memory with the signal stream information; and
(e) generating a dashboard for display to the user, the dashboard comprising graphical representations of one or more of the data analysis streams, the user insights, and the system insights.

18. The computer-implemented method of claim 17, wherein the time-series results data relating to the user comprises one or more aspects of the user’s performance of an activity, user’s health, user’s wellbeing, user’s behavior, or user’s surroundings.

19. The computer-implemented method of claim 17, wherein the signal stream information is generated from privacy-filtered input data and analyzing the time-series input data comprises generating privacy-filtered input data by:
(i) removing identifiable data from input data received from one or more of the interface devices;
(ii) extracting non-identifiable data from input data received from one or more of the interface devices; or
(iii) analyzing input data received from one or more of the interface devices to generate non-identifiable metadata.

20. The computer-implemented method of claim 17, further comprising:
(f) generating a first feedback score for display to the user based on an analysis of at least two of the plurality of data analysis streams;
(g) determining one or more recommended actions that the user can take to improve the first feedback score; and
(h) presenting the first feedback score and the recommended actions in the dashboard.

21. A system for improving user performance in one or more activities, comprising:
a plurality of interface devices communicatively coupled to and/or integrated with a user device, wherein one or more of the plurality of interface devices comprise a keyboard device, a camera device, a mouse device, a microphone, or a gaming controller;
one or more applications stored in memory, wherein the one or more applications are configured to:
(a) receive time-series input data from the plurality of interface devices;
(b) analyze the time-series input data to generate signal stream information comprising a plurality of data analysis streams, wherein one or more of the data analysis streams contain time-series results data characterizing an aspect of a user’s performance of an activity on the user device and one or more of the data analysis streams contain time-series results data characterizing an aspect of the user’s behavior during performance of the activity;
(c) receive user insights describing one or more events, ambient conditions, behaviors, mental states, and/or physical states experienced by the user during performance of the activity; and
(d) generate a dashboard for display to the user, the dashboard comprising graphical representations of one or more of the data analysis streams and the user insights.

22. The system of claim 21, wherein one or more of the applications are stored in memory of a platform device communicatively coupled to the user device and one or more of the plurality of interface devices.

23. The system of claim 22, wherein one or more of the applications are stored in memory of a peripheral device communicatively coupled to the platform device, and the dashboard is displayed to the user by use of the peripheral device.

24. The system of claim 22, wherein the platform device is integrated with one of the plurality of interface devices.
